(19) United States
(12) Patent Application Publication
(10) Pub. No.: US 2026/0030748 A1
(43) Pub. Date: Jan. 29, 2026

(54) ANALYSIS OF ANATOMICAL SURFACE TOPOLOGY AND SURGICAL GUIDE DEVELOPMENT BASED THEREON

(71) Applicant: Paragon 28, Inc., Englewood, CO (US)

(72) Inventors: Donald HUME, Denver, CO (US); Mathew Richard ANDERLE, Greenwood Village, CO (US); Laura Zagrocki BRINKER, Lone Tree, CO (US)

(73) Assignee: Paragon 28, Inc., Englewood, CO (US)

(21) Appl. No.: 19/274,936

(22) Filed: Jul. 21, 2025

Related U.S. Application Data
(60) Provisional application No. 63/675,313, filed on Jul. 25, 2024.

(51) Int. Cl. G06T 7/00; G06T 7/11; G16H 20/40

(52) U.S. Cl. CPC G06T 7/0012; G06T 7/11; G16H 20/40; G06T 2207/30008

(57) ABSTRACT

Surgical guide development based on anatomical surface topology analysis includes determining surface shape variability of a surface of bony anatomy in a region of interest, generating, based on the surface shape variability, a digital guide design of a proposed surgical guide for use against the bony anatomy during a surgical procedure, the generating determining placement and surface coverage for the proposed surgical guide and configuring the proposed surgical guide to have a corresponding footprint and guide features configured to interface with selected features of the surface of the bony anatomy, rating the proposed surgical guide for use against the bony anatomy during the surgical procedure by performing, using the digital guide design, an automated topology-based stability assessment to produce a performance rating for the proposed surgical guide, determining whether the performance rating for the proposed surgical guide satisfies a predefined performance rating threshold, and performing processing based on that determination.

### CROSS-REFERENCE TO RELATED APPLICATION

This application claims the benefit of U.S. Provisional Application No. 63/675,313, filed 2024 Jul. 25 and entitled “ANALYSIS OF ANATOMICAL SURFACE TOPOLOGY AND SURGICAL GUIDE DEVELOPMENT BASED THEREON”, which is hereby incorporated herein by reference in its entirety.

### TECHNICAL FIELD

The invention pertains to development of surgical guides, and more specifically to analysis of anatomical surface topology and development of surgical guides based thereon.

### BACKGROUND

Surgical guides are physical, often single-use, devices temporarily placed against patient anatomy to aid a surgeon or other actor in executing surgical actions relative to the patient anatomy as part of a surgical process. For example, surgical cutting guides (“cut guides”) are placed against bone to facilitate and guide accurate cuts made to the bone. Surgical guides may be patient-specific in that they can be created with patient-specific anatomical features in mind.

It is desirable when creating a patient-specific surgical guide or other patient-specific instrumentation to construct the guide to reference anatomical landmarks on the bone that are adjacent, or in near proximity to, a target site of a surgical action, such as a cut in the example of a cut guide. The guide can be built to include features that contact these landmarks and help “seat” the guide in the proper location, thereby helping to ensure that the surgical actions, such as cutting, are performed at the proper anatomical locations. For example, correct incisions, and therefore guide placement, are critical to the reliability and success of the patient-specific surgical process.

### SUMMARY

Patient-specific instrumentation, for example surgical guides, such as cut guides, may be constructed with features (“guide features”) configured to contact features of patient anatomy (“anatomy features”) in the proximity of the planned surgical actions, for instance cuts. In the context of a patient-specific cut guide, the particular location of a desired cut can present challenges to constructing a guide that reliably ensures adequate seating against the anatomy. Further, some guides might be fabricated to include common guide features intended to reference similar anatomical landmarks across different patients for a given procedure. Such a ‘one-size-fits-many’ approach can lead to unsatisfactory results. Different patients often have differing anatomical features and characteristics thereof, for instance more or less pronounced surface ridges, valleys, and other features, or anatomical alterations from prior procedures, as examples. This presents challenges due to the absence of unique bony conformity in the surgical guides used and/or spacing of the location of the surgical actions relative to a traditional anatomical feature. Even reuse of guides intended to seat against relatively featureless anatomy (such as smooth cylindrical or flat surfaces) can present difficulties. As a result, it can be difficult to ensure that a patient-specific surgical guide will interface well with the surface of the bone against which it is intended to sit.

Shortcomings of the prior art are overcome and additional advantages are provided through the provision of computer-implemented methods.

In an embodiment, a method includes determining surface shape variability of a surface of bony anatomy in a surgical region of interest. The method further includes, based on the surface shape variability, generating a digital guide design of a proposed surgical guide for use against the bony anatomy during a surgical procedure, the generating determining placement and surface coverage for the proposed surgical guide and configuring the proposed surgical guide to have a corresponding footprint and guide features configured to interface with selected features of the surface of the bony anatomy. The method additionally includes, rating the proposed surgical guide for use against the bony anatomy during the surgical procedure by performing, using the digital guide design, an automated topology-based stability assessment to produce a performance rating for the proposed surgical guide. Additionally, the method includes determining whether the performance rating for the proposed surgical guide satisfies a predefined performance rating threshold, and performing processing based on whether the proposed surgical guide satisfies the predefined performance rating threshold.

Additional aspects of the present disclosure are directed to systems and computer program products configured to perform the methods described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIGS. 1A-1F depict example digital representations of anatomy surface shape variability, in accordance with aspects described herein;

FIG. 2 depicts an example contour plot of patient anatomy with a proposed guide footprint for feature interaction, in accordance with aspects described herein;

FIG. 3 depicts an example process flow for developing a patient-specific surgical guide and quantifying its stability score using an algorithmic workflow, in accordance with aspects described herein;

FIG. 4 depicts an example interface for calculating and conveying surface stability metrics and exporting a contour plot file for secondary usage, in accordance with aspects described herein;

FIG. 5 depicts an example process for surgical guide development based on anatomical surface topology, in accordance with aspects described herein; and

FIG. 6 depicts an example computing system/environment to incorporate and/or use aspects described herein.

### DETAILED DESCRIPTION

Described herein are aspects and technology to facilitate design, testing, and fabrication of patient-specific surgical guides (also referred to herein as patient-specific guides or instrumentation). Topology-based stability determinations are used for patient-specific instrumentation (PSI) guide design. For instance, some aspects identify anatomical features of a patient anatomical region that are most optimal (or conversely least optimal, i.e., for the sake of avoidance) for a patient-specific surgical guide to reference in order to reliably seat the guide against the anatomy and thereby help ensure proper positioning for desired surgical actions. Aspects can be provided as part of a production workflow with algorithmic implementation to better inform guide shape, location, and placement. Many concepts and examples discussed herein are presented in the context of a surgical cut guides, though this is by way of example and not limitation; aspects discussed herein apply to other types of surgical guides/instrumentation, for instance drill guides, burr guides, alignment guides, target guides, and instrument guides, among others.

In embodiments, the fit of a patient-specific surgical guide is optimized by analyzing the surface topology of target anatomy, for instance a bone to be cut, and possibly adjacent anatomy, using the patient-specific surgical guide. ‘Fit’ in this context can include various aspects such as accuracy in terms of placement, matching of the bone surface, and other measures. Optimizing the fit of the guide will help a user, such as the surgeon, identify and properly position/seat the guide against the bone prior to pinning it to the bone. Proper location for pinning will help ensure that the surgical action (e.g., cut) when executed will be made at that intended position.

Process(es) executing on computer system(s) can perform aspects described herein, for instance to acquire input data, such as digital image data from imaging equipment, and perform processing to generate a digital guide design of a patient-specific guide. The design can be reviewed, tweaked if desired, and provided to a fabrication device for guide fabrication, e.g., printing. The process can be overseen by a user, for instance a PSI or development engineer, if desired.

An example process examines a scan or other input digital image data indicative of surface properties, such as a surface topology, of a surface of bony anatomy of interest, and quantifies the deviations in surface topology along the surface. For example, input image data is segmented to extract a digital representation of the bony anatomy with a specific focus on the outer surface of the bony anatomy (“surface” in this context may refer to a collection of surfaces, or portions of surface(s), of one or more bones). The surface is then analyzed by way of the digital representation to quantify the variability in surface shape (“surface shape variability”), e.g., changes in the surface relative to respective area(s) across a surface range. The range may extend, as an example, from no change in surface anatomy (e.g., completely flat and planar) to a relatively drastic change in surface anatomy (e.g., a tall protrusion with a small cross-sectional area). The process can identify areas on the bone surface that have relatively high surface shape variability, for instance relatively large deviations (e.g., a bump, ridge, crest, etc.) in surface topology per unit of area and/or areas on the bone surface that have relatively low surface shape variability, for instance relatively minimal deviations in surface topology per unit of area. A given area with little or no surface deviation is not expected to contribute significant or sufficient stability for seating the guide against the bone. The quantified deviations in surface topology can therefore influence guide design and also inform how well a created guide is expected to work for a given patient and procedure. In designing a guide, parameter(s) in addition to maximizing the stability of the guide may be considered, for instance guide size, features that must or must not be included due to anatomical limitations, etc., The process can quantify the expected performance of the guide in one or more areas to provide a performance rating or score for the guide, which can be compared to a predefined performance rating threshold. Such a threshold or other quantitative measure can be used to evaluate the stability of the proposed guide for a particular application, i.e., assess and determine whether a given propose surgical guide is suitable for a given patient to undergo a given surgical procedure using the proposed guide. The process can provide any desired output to a PSI or development engineer. Example such outputs include model(s) or other representations of the surface anatomy (and/or variability thereof) of the bony anatomy, a model of the surgical guide for evaluation, the performance rating for the proposed guide, and ultimately, a fabricated surgical guide.

Thus, in one aspect, a process receives various inputs, for instance digital image data input. The imaging data is directly or indirectly (e.g., based on further processing) indicative of surface properties and topology of patient-specific bony anatomy in a surgical region of focus for the procedure, referred to as the surgical region of interest (ROI). By way of non-limiting example, the digital image data is computed tomography (CT), weight-bearing CT, x-ray, weight-bearing x-ray, magnetic resonance imaging (MRI), or ultrasound-based imaging data of an anatomical ROI. In some embodiments, the image data could be in the form of a digital three-dimensional (3D) model created from other imaging data, such as one or more x-rays.

In any case, after receiving the patient-specific digital image data, the process can extract the bony anatomy in the surgical ROI, for instance by segmenting the digital image data if needed. Such acquisition of image data and segmentation thereof may not be necessary if a segmented bony geometry/surface model for the ROI is already available as input to the process.

Additional input to the process can include indications of potential properties or constraints to be considered in guide design. Such additional inputs might inform parameters for guide size, anatomy to be avoided (e.g., soft tissue), and other constraints. For instance, a maximum incision size to access the region of interest might dictate a maximum size of the guide.

The process then analyzes the bony anatomy—specifically the digital representation of the surface topology of the surface of the bony anatomy—to calculate surface shape variability/deviation. Initially, surface shape variability (topological variation) of the surface is assessed and determined for discrete points/areas of the anatomy surface. The calculation may be geometry-independent and performed for any number of bones and joints. The calculated surface shape variability at these discrete points (there might be a respective indication of variability corresponding to each point, or one or more indication(s) corresponding to a collection of points) can be used to identify regions of high (and/or low) shape variability. Different modes of shape variability may be used, for instance principal curvature, first and/or second derivatives, and/or frequency response, as examples.

To help illustrate, consider a distal tibial osteotomy (also referred to as a supramalleolar osteotomy, or SMO) procedure. The process could analyze, in this example, the surfaces of the distal tibia and distal fibula. It is noted that at this point bone density of the bone or other features of the bony anatomy (other than the surface topology) need not be considered, though these features could be considered at some point in the process if desired, as they could influence screw/pin/implant placement, and therefore guide stability. The process calculates surface shape variability using, e.g., one or more of principal curvature, first and/or second derivatives (f′, f′), and frequency response of shape curvature, at discrete points along the bony surface. The shape variability at these points can be applied to the PSI design process as described herein to better inform guide properties, for instance guide footprint, placement, and shape.

In some embodiments, more than one of the numerical approaches mentioned above (principal curvature, f′, f″, frequency response) may be combined/composited to define the shape variability. In this regard, there is an opportunity to tune the numerical representation of shape at that surface point to best inform stability properties of that point with respect to the desired surgical application (guide/procedure specific cost function).

An example of a combination of numerical approaches for defining shape variability is as follows:

Iniitally, define the following terms:
C₍ₙ₎: the principal curvature calculated at a singular node n, of N total nodes
wₑ: the weighting factor for the principal curvature calculation in the overall cost function
F₍ₙ₎: the topology frequency response calculated at a singular node n, of N total nodes
w_{f}: the weighting factor for the frequency response calculation in the overall cost function

An example combined and weighted cost function (CF₍ₙ₎) using multiple metrics calculated at each node n (composite score) is given by:

$${CF}_{( n )} = {w}_{c} * {C}_{( n )} + {w}_{f} * {F}_{( n )}$$

This equation is a cost function for a given point/area identified at node n (with coordinates [x,y,z]) on the surface, and determines the shape variability for that specific point (x,y,z). The above provides a linear representation including weighted terms which would be defined based on their overall contribution to the cost function (CF). In other words, each of the weighting factors can be set individually to establish the contribution of the corresponding term to the cost function. The CF could also have increased degrees of freedom, such as a polynomial with 2^{nd} and 3^{rd} order terms. The weights wₑ and w_{f} could be tuned to create an optimal response.

In examples, the cost function overall could include components that influence both “grip” and “stability” assessment:

Grip—informed by a unit area which has a high change in principal curvature. Example: A relatively large dimple (anatomical feature) exists on the bone surface, and a 1 millimeter (mm)×1 mm guide footprint feature could interface and “grip” with this anatomical feature. Numerically, this anatomical feature would have a high curvature and show as a good grip metric.

Stability—If a 1 mm×1 mm footprint feature, which is relatively small, exists on the surgical guide even with a large curvature feature under the footprint, the guide might wobble, rotate, and be unstable on the bone due to the small surface area. Another piece of the cost function could therefore include stability and other degrees of freedom. One example of an important factor might be a percent of cross-sectional circumference (‘wrap’).

In general, finding an optimal or more desired solution may not be as straight-forward as finding a relatively small surface area on the bone that has large magnitudes of curvature; there may be other appropriate factors related to stability/grip, such as coverage, shape, and bony interaction.

The surface shape variability assessment continues by interpolating, from the determined surface shape variability at the discrete points of the surface, surface shape variability across at least a portion of the bony anatomy surface to determine the surface shape variability of the surface. As an example, variability at some of the discrete points mentioned above is used to interpolate surface shape variability at other discrete points of the surface. The collection of shape variabilities (actual and interpolated) can then be used to build visualization(s) that highlight surface variability for the surface of the bony anatomy, including regions of high and/or low variability. For instance, the process generates and displays, on a display device as part of an anatomy model of the bony anatomy, a digital representation of the surface shape variability of the surface.

Consider an example in which a substantially cylindrical can representing a bony anatomy (for instance a tibia) has round outer walls and top and bottom lips, and that a desired action is to make a cut that will cut the can in half horizontally. It is desired to make a patient-specific surgical guide (a cut guide in this example) for this cut, and the guide is to reference features on the can near where the cut is to occur that will help stabilize the guide to help ensure that the cut slot remains in the desired location at the halfway point on the can. If the algorithm to quantify the surface of the bony anatomy based on its capability to aid in stabilizing the patient-specific cut guide were applied across the surface of the can, the round outer walls of the can would score relatively low because there are not many features that could be added to the guide that will interface with the round wall of the can in a way that would increase stability of the guide. However, the top and bottom lips of the can would score relatively high because they could serve as ‘anatomical features’ with which guide features of the guide could interface/engage. For instance, guide features, such as arms with hooks, could be added to the guide to interface with these ‘anatomical’ features of the can and add stability to the guide when interfaced with the can.

Optimal regions of the surface of the bony anatomy for interfacing with stabilization features of the guide can, in some examples, be those regions of the anatomy having relatively high shape variability within a relatively concentrated (small) area. Elevational changes such as ridges and grooves, corner-like features, and other anatomy features may all be good features to serve as bases with which corresponding guide features can be configured to interface for stability.

Once the shape variability of the anatomy has been scored, the process generates the digital representation, which could include one or more of any number of representations of this scoring. The representation can convey, indicate, highlight, etc. region(s) of the anatomy having higher and/or lower surface shape variability relative to other region(s) of the surface. An example output digital representation is a heat map anatomy model of the bony anatomy, in which each of various areas of the surface are scored on a scale, for example a scale of 0 to 1, with 0 being least optimal and 1 being most optimal. A corresponding color (or other informative) coding may be used over the entirety of the anatomy. By way of specific example using a color-based representation, green or blue color could be used for areas scoring closer to 0 and red or yellow could be used for areas scoring closer to 1. A color spectrum could be used with a corresponding color on one end representing a score of 0 and another color on the other end representing a score of 1, with colors between these two extremes being used for corresponding areas between the two scores.

An engineer could analyze the anatomy model based on the color-coded representation and select surface features that a guide could reference for engagement. Additionally or alternatively, the algorithm could flag or otherwise identify regions/areas with relatively high (or low) surface shape variability to indicate areas to focus on or avoid, respectively, in terms of stability reliance.

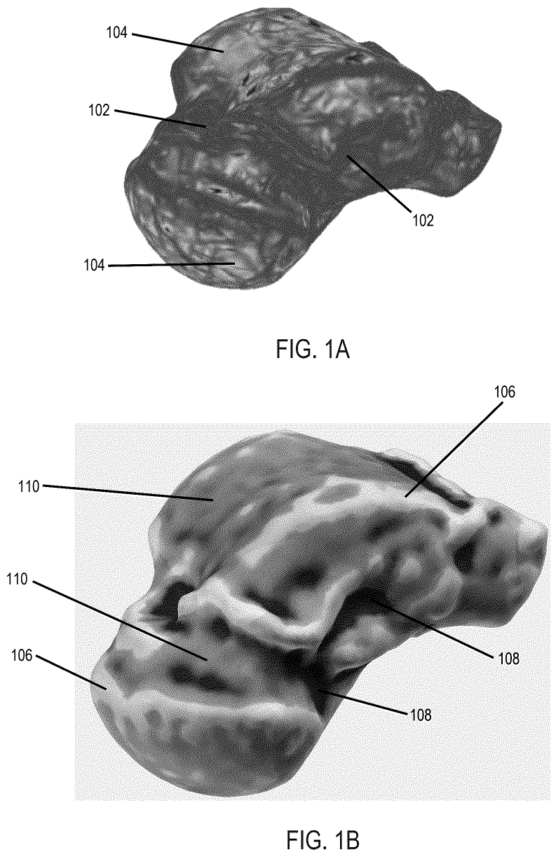

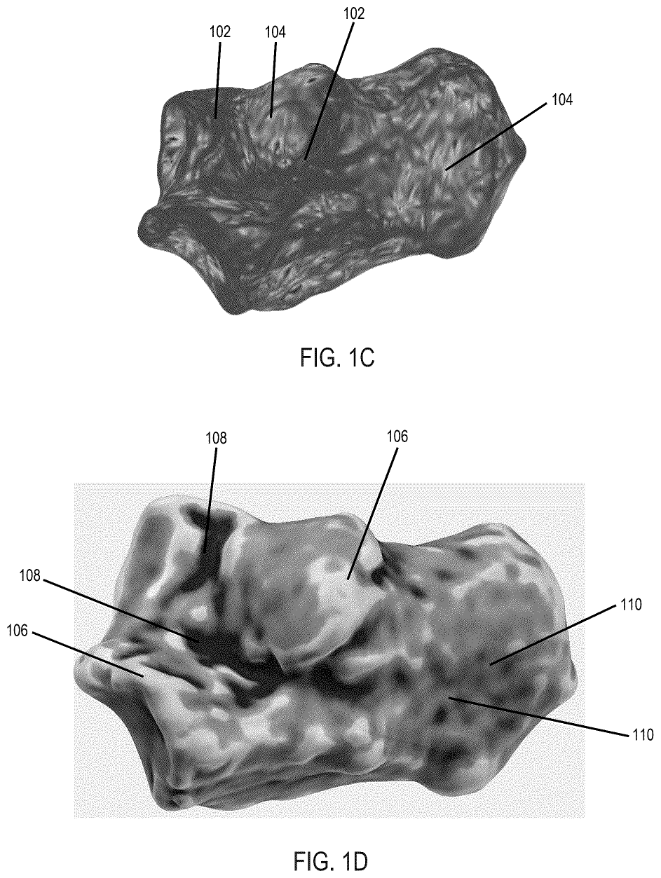

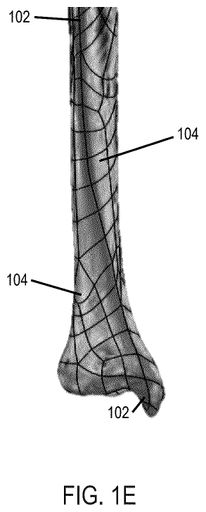

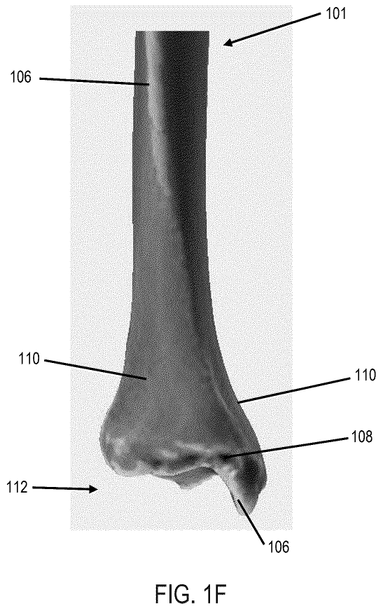

FIGS. 1A-1F depict example digital representations of anatomy surface shape variability in the form of heat map anatomy models. FIGS. 1A and 1B depict example such anatomy models for a talus bone, FIGS. 1C and 1D depict example such anatomy models for a calcaneus bone, and FIGS. 1E and 1F depict example such anatomy models for a tibia bone. FIGS. 1A-1F, provide 3D visual representations in the form of contour plots illustrating the variability metrics (numerical scoring) for the depicted bones/ROIs, including illustrating regions of differing interest and stability in varying grayscale colors. In FIGS. 1A, 1C & 1E, a grayscale spectrum varying from relatively dark to relatively light is used, in which areas of relatively high magnitude of positive curvature are shown in relatively dark grayscale (as exemplified at points 102), and areas of relatively low magnitude of curvature are shown in relatively light grayscale (as exemplified at points 104). In FIGS. 1B, 1D & 1F, areas of relatively high magnitudes of positive and negative curvature are shown in relatively light grayscale (as exemplified at points 106) and relatively dark grayscale (as exemplified at points 108), respectively, and near-zero curvature across the surface is shown in moderate grayscale (as exemplified at points 110) between relatively light and relatively dark. Referring to FIG. 1F by way of example, it is seen that the far end 112 of the tibia 101 has a lot of variability in shape at the base and a ridge on an interior edge of tibia.

With the anatomy scored, an engineer or automated process can use this output to generate a digital guide design of a proposed surgical guide for use against the bony anatomy during a specific surgical procedure. This can include determining placement and surface coverage for the proposed surgical guide and also configuring the proposed surgical guide to have a corresponding footprint and guide features that are configured to interface with the selected surface features.

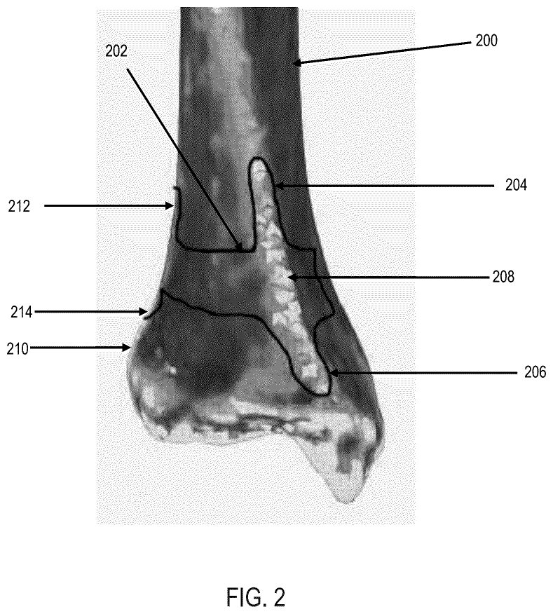

FIG. 2 depicts a contour plot for a tibia 200 illustrating high-stability regions of interest (in relatively light grayscale; based on curvature) together with a proposed guide footprint 202 shown as an outline for feature interaction between the anatomy and the guide. The guide footprint 202 includes legs 204, 206 extending vertically up a first ridge feature 208 of the tibia 200. The footprint 204 extends horizontally toward a second ridge feature 210 (partially depicted) around the left side of the tibia 200, and has corresponding legs 212, 214 extending vertically for engagement with the second ridge feature 210.

A digital model/design of the guide could be created and configured to have and conform with the corresponding footprint, including guide features that are configured to interface with the optimal portions of the surface (and potentially avoid the suboptimal portions) as reflected by the footprint, and comport with any applicable constraints on guide properties, like guide size for instance. In this regard, a user could input whatever constraints/parameters are desired. Aspects of the configuration could also account for other properties that are to be considered but perhaps not necessarily maximized (like is desired with stability). For example, constraints can be set to avoid soft tissue structures near the bony anatomy, or to maximize one or more dimensions of the guide so as to limit or minimize a required incision size to properly place the guide.

In some specific examples, generating the digital guide design of the proposed surgical guide uses a partially or wholly automated footprint generation process that uses a footprint growing algorithm that grows a footprint of the proposed surgical guide by prioritizing and selecting anatomy surface features, based on the shape variability, to dictate where the footprint is to extend (e.g., outward from a starting point, for instance) and which guide features to incorporate in the proposed surgical guide in order to effectively engage with those selected surface features.

The proposed surgical guide as modeled could then be rated for use against the bony anatomy for the surgical procedure by running the guide design through an automated topology-based stability algorithm that produces a numerical performance rating relative to the given anatomy (such as that reflected by the input imaging data above). In this regard, the initially-modeled guide could be in a ‘draft’ form that is subject to modification. The guide performance rating reflects a stability metric specific to that guide and that given anatomy for which it was modeled. The performance rating can be wholly or partially based on the proposed footprint of the guide relative to the patient-specific anatomy against which is proposed to be used, and can take into account the specific surgical procedure. Furthermore, the rating could be based on any number of different factors whose objective is to provide a performance criterion that informs engineering on good and/or poor designs. A process could compare the performance rating for the draft guide to a predefined performance rating threshold to see whether the performance rating satisfies the threshold, for instance meets (or exceeds) a minimum threshold number, or does not exceed a maximum threshold number, as examples. This determination could yield a pass/fail score to the guide design, in which a ‘pass’ moves the process to the next step and a ‘fail’ enters an iterative feedback loop for the system and/or engineer using the process. Processing can proceed accordingly. For instance, in ‘fail’ situations, the process could modify the guide design by modifying properties of the proposed surgical guide. The modifications could be accomplished by an engineer iterating on modifications of the guide design, as an example T process can re-rate the proposed guide with the modified design, and iterate on this until the performance rating satisfies the threshold. Once the guide design, modified as needed, passes and is approved, the guide design can be provided for fabrication for use during the intended procedure and fabricated.

In some examples, the performance rating algorithm can be built based on feedback obtained for existing, prior-used guides and their effectiveness for given procedures against prior anatomies. A guide used during a surgery could be assigned a numerical value corresponding to whether the guide worked effectively during that surgery for that specific prior anatomy involved and/or corresponding to a degree of effectiveness for that surgery and specific prior anatomy involved. For instance, a 0 or 1, or some scale or spectrum of values could be used to indicate the effectiveness/appropriateness of the given prior guide for the given prior procedure on a given prior patient. Similarities between a prior-used guide and the draft guide being developed, and/or between the prior anatomy and the current patient-specific anatomy, could be assessed to determine an expected performance of the draft guide on the current patient-specific anatomy. Artificial intelligence (AI) could be used to score a proposed guide relative to performance of other guides known to have ‘passing’ ratings and/or some minimum (excellent) stability. This could be facilitated by training an AI model to identify and score certain features on the proposed guide relative to the current anatomy based on how similar features performed on similar prior anatomy in other cases, with the scores of these various guide features on the proposed guide then being aggregated to generate the overall score of the proposed guide.

Additionally or alternatively, a process could use computer software to perform a computational analysis on the guide design, with the computational analysis assessing stability of the guide for the current patient-specific bony anatomy during the procedure, and with the performance rating being a function of the results of that computational analysis. One example computational analysis that may be effective is rigid body analysis, though others are possible.

In terms of the stability metric, each area (e.g., point or collection of points to define a discrete area) of several areas on the bone surface can be associated with a respective score and be assigned a color (or other representation) associated to the score for that area. One approach for determining the performance rating of the proposed guide could be to take all of the scores for each point/area covered by the guide footprint and average them as a function of surface area of the guide design to determine a numerical representation. This could then be compared against a pre-determined threshold to see whether the determined performance rating meets or exceeds the threshold.

A threshold could be set based on targeted feedback that is gathered from actual use of guides in the field, however this need not necessarily be the case. For instance, the threshold could be determined based on some other metrics, such as existing knowledge about which guides historically have, or have not, performed well for given surgeries. Internal testing could also be used to determine an appropriate threshold to use.

In some embodiments, the threshold is determined as a weighted calculation of terms. For instance, the weighted calculation could take into account a composite (e.g., average) surface area score for that guide, and optionally procedure-specific parameters/terms. Requirements or preferences related to guide size could be weighted to influence the threshold used and/or performance rating of a given guide. For instance, different requirements or parameters in the form of measurements of a requisite (e.g., maximum) incision size, such as 3″, 2.5″, etc., with smaller being generally more preferred, to accommodate the guide could be weighted differently than each other. A larger incision size required for a larger guide footprint might decrease the performance rating, but this might be outweighed if significantly better stability is achieved in having the slightly larger guide footprint, for instance.

From a purely numerical standpoint, the performance rating for a guide is expected to be bone-specific, location-specific, and perhaps procedure-specific. In other words, the performance rating of a given guide design may be influenced by (at least) the properties of the specific bone against which is proposed to be used, the location for that guide on the bone, and the procedure for which it is proposed to be used. Placing a given guide on a different bone or in a different region of the same bone will result in coverage over different surface areas with different scores, and is very likely to lead to a different performance rating.

Regarding the procedure involved, the performance ratings and thresholds used to determine guide pass/fail for use might vary based on procedure because of the specific bones involved and bony areas against which the guides are to sit. For instance, it might be difficult to achieve a high guide performance rating for a particular procedure that requires a relatively small guide against a bone surface that is typically relatively featureless. The performance rating threshold might be set relatively low in these cases. Alternatively, a scale being used for rating/thresholding could be normalized so that an ‘A’ grade or ‘pass’ on one procedure, such as a Lapidus procedure, requires significantly greater stability than an ‘A’ grade or ‘pass’ on another procedure, as an example.

Aspects can also include other elements of automation. For example, a system may be able to take a digital guide template as a starting point, e.g., initial digital guide design for a proposed surgical guide to be designed/fabricated, and, based on the most and/or least optimal portions of the surface of the target patient-specific bony anatomy, tweak the initial digital guide design based on patient-specific anatomy features reflected by the surface shape variability. The tweaking can be geared to impart guide features isolating (engaging with) the most optimal anatomical features/portions of the patient-specific anatomy, and optionally avoid the least optimal anatomical features/portions of the anatomy. This could then be reviewed by an engineer or physician for approval.

In a specific example, a fitting algorithm could be used with an average anatomy shape of a population to develop a ‘nominal’ guide template design. A statistical shape model—SSM—of the average shape of the bone/joint of the population could be determined, along with the primary modes of variation in that anatomy across that population, to build a SSM and indications of how that SSM might vary. The average bone geometry as informed by the SSM can be input to the topology-based surface shape variability determination discussed above, and the output can be used to develop the ‘nominal’ guide template design. In this regard, an ‘optimum’ guide design for the average anatomy is determined as a good template with which to begin, before tailoring the design based on patient-specific anatomy. When a patient-specific anatomy is input, a process can utilize the ‘nominal’ digital guide template as an initial guide design, recommend updates to this design, i.e., changes to ‘nominal’ design, which changes might be informed by the primary modes of variation as determined above, to inform an optimal PSI guide design and assist an engineer or other user in visualizing optimal guide location. Thus, with an understanding of the average shape of a population, the process can develop a baseline digital guide template design. Then, the patient-specific process proceeds as described above except that instead of, or in addition to, providing the heatmap or other representation of the patient-specific surface anatomy scores, the process can recommend changes to what is reflected by the template in order to move the nominal design toward a patient-specific design, for instance by analyzing how the patient-specific bony anatomy compares to the nominal bony anatomy, then using patient-specific features to tweak the footprint (and optionally other features) of the guide informed by the template to comport with the patient's specific surface anatomy and produce the patient-specific guide design.

The patient-specific guide design might be taken as ‘optimal’ in terms of providing the best footprint and stability based on the provided constraints (surgical window, amount of bone accessible, etc.). However, the engineer or other user might desire an opportunity to examine/visualize the ‘optimal’ guide in terms of footprint or other properties and make changes. The user might want to impart other constraint(s) or tweak the guide if it does not significantly reduce the performance rating, as an example. Accordingly, the engineer (or other user) could iterate on this ‘optimal’ design. The engineer might want to iterate on the design for other reasons, for instance if the design is still deemed too poor to use, or a pass threshold was not used or was too low.

Guide designs other than ‘nominal’ designs formed based on an average population could alternatively be used as starting points for designing a new patient-specific guide. For instance, software to perform processing as described herein can learn and confirm over time the guide designs that fit well with certain anatomies. When the software recognizes that a new patient-specific anatomy is similar to a prior anatomy, the software can identify a guide design that fit well with the prior anatomy, and use that identified guide design as a starting point for designing a guide for the new anatomy. This could also be expanded to the SSM-based designs discussed above; there could be a collection of multiple different ‘nominal’ digital guide templates corresponding to different sub-populations of a larger population. One guide template design might be for the overall average, another guide template design might be for anatomy within two standard deviations above of the average along a given dimension, and a third guide template design might be for anatomy within two standard deviations below the average along the given dimension, for instance. Any number of nominal digital guide templates could be established as potential starting points, and built based on population average anatomies of discrete populations. A new patient-specific anatomy could be compared to the population average anatomies to which the templates correspond. Similarities between the patient-specific anatomy features and a given population average anatomy could inform the digital guide template to use. This finds the template to which the patient-specific anatomy most closely matches, and the process could use the design informed by that template as the starting point for further tailoring to the patient's specific anatomy. Using the example of FIG. 2, maybe a sub-population does not have as pronounced a ridge 208, and therefore the starting nominal guide design is selected to be one that works best with the average for the sub-population lacking such a pronounced ridge 208.

Population modeling and baseline/nominal guide designs could, in some examples, be packaged into existing software for PSI guidance and planning. The software could use the topology-based stability metric, combined with location optimizations constrained by bone shape, surgical technique, and parametric design constraints (as examples) on a PSI guide model. In these examples, an SSM representation of anatomy and corresponding nominal designs could exist within a software package that is used to obtain imaging data for a patient-specific anatomy, automatically segment the anatomy and create a model of the patient-specific bony geometry, provide optimal guide properties based on results of the stability algorithm, report performance rating of that guide with respect to the threshold, and enable a user to view a parametric 3D model design of the guide for the intended surgical procedure and make changes as desired. Any such changes could prompt the software to determine and provide real-time updates to the stability metrics, performance rating, and 3D design geometry.

During surgery, it is common for a surgeon to place the guide against the anatomy in an initial position and then move the guide around until the surgeon feels some physical feedback, like the guide ‘clicking’ into place. Aspects can help to avoid situations where a surgeon misplaces a fabricated guide into a ‘false home’ that ‘feels’ like the correction position. For instance, a process can examine the footprint of a guide design and perform a surface fitting across other parts of the bone to see the differences between the guide's intended placement (optimal surface coverage) and unintended placement on other parts of the bone. The process could provide a printout that illustrates the true position for guide versus any potential ‘false home’ positions. Additionally or alternatively, the stability metric could help inform a ‘movement’ for how a user should apply the guide to the bony surface to properly position it into place. For instance, the process could provide a specific surgical ‘application’ of the guide (including directionality of applied force, for example rotation clockwise or counterclockwise, as an example) which seeks to find the home/position for the guide in an efficient manner based on localized surface features relative to features of the guide.

Guides created using the processes described herein offer more reliable cuts intraoperatively due to greater stability in comparison than guides produced under current practices and/or with standard features that are not tailored to the specific patient.

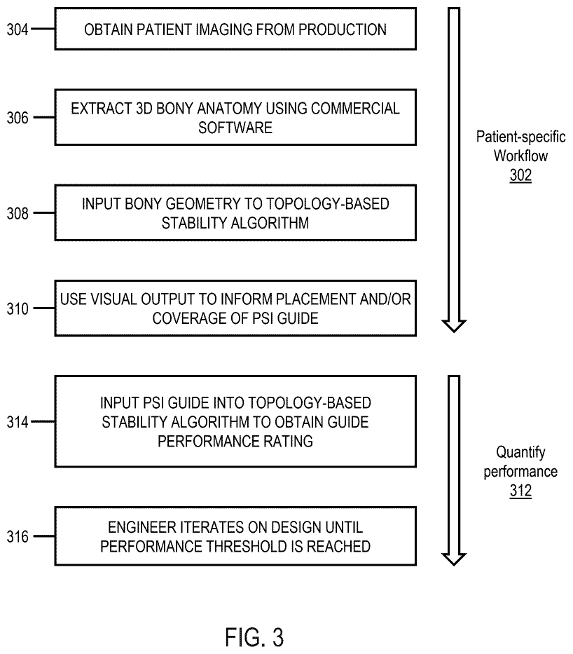

FIG. 3 depicts an example process flow for developing a patient-specific cutting guide and quantifying its stability score using an algorithmic workflow, in accordance with aspects described herein. The process could be performed at least partially by software executing on one or more computer system(s), as an example. A patient-specific workflow aspect 302 includes obtaining (304) patient imaging. Example patient imaging is or includes computed tomography (CT) scan(s). Aspect 302 also includes extracting (306) a 3D geometry of patient bony anatomy reflected by the patient imaging, and inputting (308) the 3D geometry to a topology-based stability algorithm as described herein. In this regard, the inputting (308) takes the bony anatomy (e.g., with no reference to an external guide), and calculates the stability score across the bone surface. This can produce an output, like that of FIG. 1B, for instance. The patient-specific workflow aspect 302 also includes receiving (310) placement/coverage of a patient-specific instrumentation (PSI) guide, for instance based on an engineer or other user using visual output of the algorithm (from 308) to inform the placement/coverage. In this aspect, the engineer can use that visual cue to design a guide that is to work well based on, e.g., the engineer's professional experience and the data provided by the algorithm for the bony surface.

Based on the patient-specific workflow aspect 302, the process flow performs quantify performance processing 312, in which the PSI guide is input (314) into the topology-based stability algorithm to obtain a guide performance rating, and the engineer or other user iterates (316) on the design until a performance threshold is reached. In an example embodiment, the inputting 314 takes the guide design (from aspect 302) and calculates the stability score for, e.g., only the footprint of the guide on the bone surface. Optionally, this could be distilled down to a singular value within a larger pass/fail threshold to give the engineer feedback about the expected stability performance of the guide created. At 316, optional iteration(s) of the design could be performed, for instance to revise or tweak the design to improve the stability score, for example. If no iteration(s) are performed, or after any desired iteration(s), the process could move forward with the design, if satisfactory.

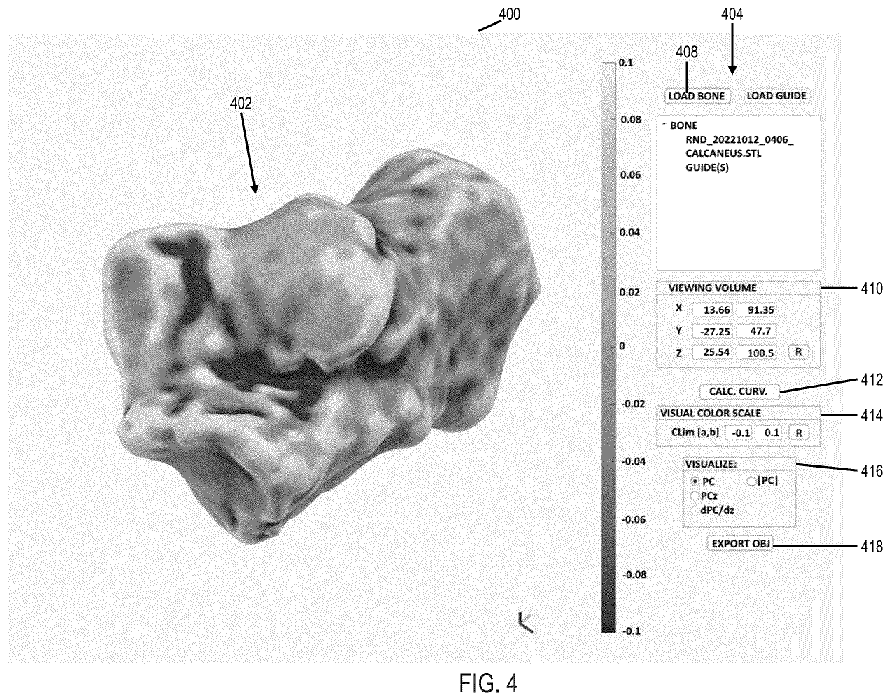

FIG. 4 depicts an example interface for calculating and conveying surface stability metrics and exporting a file, for example a colored geometry file (as an .OBJ file, for instance) for secondary usage, in accordance with aspects described herein. Interface 400 can be built and presented on one or more display devices, for instance display devices of a computer system supporting the engineering and design of a patient specific surgical guide. The interface could be presented to an engineer and/or other user(s). The interface presents a bone model 402 of patient anatomy as a contour plot reflecting stability metrics using a visual color scale, properties of which are set/selected in a model properties portion 404 of the interface 400. The model properties portion 404 has an interface element 408 for selection to load a bone model (by way of file selection/specification, for instance), a viewing volume properties element 410, a calculate curvature element 412 (e.g., as a button) to trigger calculation of surface curvature of the modeled bone, visual color scale properties element 414 for setting the visual color scale to use, a Visualize selection area 416 for selection among available option(s) for visualization, and ‘export as file’ element 418 for selection to export the colored contour plot as, in one example, a virtual geometry file (for instance an OBJ file).

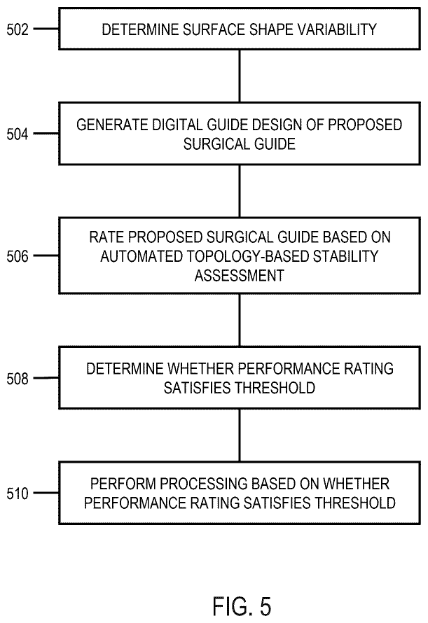

An example process in accordance with aspects described herein is depicted and described with reference to FIG. 5. The process determines (502) surface shape variability of a surface of bony anatomy in a surgical region of interest, and further, based on the surface shape variability, generates (504) a digital guide design of a proposed surgical guide for use against the bony anatomy during a surgical procedure. The generating 504 determines placement and surface coverage for the proposed surgical guide and configures the proposed surgical guide to have a corresponding footprint and guide features configured to interface with selected features of the surface of the bony anatomy.

In embodiments, the surgical guide is a cut guide.

In some examples, determining the surface shape variability 502 of the surface includes determining surface shape variability at a plurality of discrete points of the surface, and interpolating, from the determined surface shape variability at the plurality of discrete points of the surface, surface shape variability across at least a portion of the surface to determine the surface shape variability of the surface. In such examples the process can further include automatically identifying discrete points, of the plurality of discrete points, as having relatively high surface shape variability compared to other discrete points of the plurality of discrete points, and selecting the features of the surface with which the guide features of the proposed surgical guide are configured to interface based on the identified discrete points.

In some embodiments, generating the digital guide design of the proposed surgical guide uses a partially or fully-automated footprint generation process incorporating a footprint-growing algorithm that grows a footprint of the proposed surgical guide by prioritizing and selecting, as the selected features of the surface, anatomy features based on the surface shape variability to dictate (i) where to extend the footprint and (ii) the guide features to incorporate in the proposed surgical guide in order to effectively engage with the selected features of the surface of the bony anatomy.

In some embodiments, generating the digital guide design of the proposed surgical guide accounts for constraints on guide properties, including guide size. In examples, at least some of the constraints on guide properties are user-specified constraints. Additionally or alternatively, in some examples, the constraints on guide properties include at least one of: avoidance of contact between the proposed surgical guide and identified portions of the bony anatomy, and a maximum dimension of the proposed surgical guide.

In some embodiments, generating the digital guide design of the proposed surgical guide selects a digital guide template as an initial digital guide design, and tweaks the initial digital guide design based on patient-specific anatomy features reflected by the surface shape variability of the bony anatomy. In examples, the digital guide template is selected from a collection of available digital guide templates. Additionally or alternatively, in some examples, a collection of available digital guide templates is built based on population average anatomies of discrete populations, and the selected digital guide template is selected based on similarities between the patient-specific anatomy features and a population average anatomy corresponding to the selected digital guide template.

Continuing with the process of FIG. 5, the process also rates (506) the proposed surgical guide for use against the bony anatomy during the surgical procedure by performing, using the digital guide design, an automated topology-based stability assessment to produce a performance rating for the proposed surgical guide.

In some embodiments, performing the automated topology-based stability assessment performs a rigid body analysis on the digital guide design that assesses stability of the proposed surgical guide for the bony anatomy during the surgical procedure, and the performance rating is a function of the assessed stability.

In some embodiments, the automated topology-based stability assessment is based on: (i) feedback from use of other surgical guides against other anatomies; and (ii) similarities and/or differences between: (a) the proposed surgical guide and the other surgical guides; and/or (b) the bony anatomy and the other anatomies.

In some embodiments, the automated topology-based stability assessment applies an artificial intelligence (AI) model trained on feedback from use of other surgical guides against other anatomies to score the proposed surgical guide for use against the bony anatomy during the surgical procedure.

In some embodiments, the automated topology-based stability assessment determines the performance rating as a function of a plurality of surface shape variability values at a plurality of points, on the anatomy surface, with which the footprint of the proposed surgical guide is configured to engage.

The process of FIG. 5 additionally determines (508) whether the performance rating for the proposed surgical guide satisfies a predefined performance rating threshold. In examples, the predefined performance rating threshold is a function of the surgical procedure for which the proposed surgical guide is proposed for use. The process then performs processing (510) based on whether the proposed surgical guide satisfies the predefined performance rating threshold.

As an example, based on the proposed surgical guide satisfying the predefined performance rating threshold, the performing processing includes providing the digital guide design for fabrication. In some examples, the process also physically fabricates the proposed surgical guide in accordance with the digital guide design.

Alternatively, in examples, and based on the proposed surgical guide not satisfying the predefined performance rating threshold, the performing processing includes modifying the digital guide design of the proposed surgical guide to modify properties for the proposed surgical guide, and re-rates the proposed surgical guide with the modified properties.

In some embodiments, the process further includes obtaining input digital image data indicative of a surface topology of the surface of the bony anatomy, and segmenting the digital image data to provide a digital representation of the surface topology of the surface. The determining the surface shape variability of the surface can be performed using the digital representation of the surface topology of the surface.

In some embodiments, the process further includes generating and displaying on a display device a digital representation of the surface shape variability of the surface. In examples, the digital representation visually indicates regions, of the bony anatomy, having higher and/or lower surface shape variability on the surface relative to other regions on the surface. In some examples, the digital representation includes a heat map anatomy model of the bony region of interest.

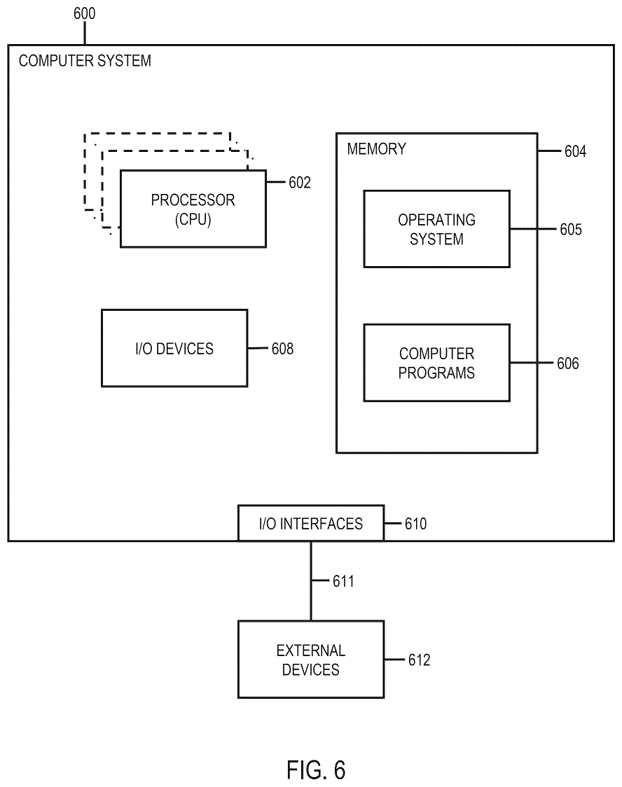

Processes described herein may be performed singly or collectively by one or more computer systems. FIG. 6 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 6 shows a computer system 600 in communication with external device(s) 612. Computer system 600 includes one or more processor(s) 602, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 602 can also include register(s) to be used by one or more of the functional components. Computer system 600 also includes memory 604, input/output (I/O) devices 608, and I/O interfaces 610, which may be coupled to processor(s) 602 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 604 can be or include main or system memory (e.g. Random Access Memory) used in the execution of program instructions, storage device(s) such as hard drive(s), flash media, or optical media as examples, and/or cache memory, as examples. Memory 604 can include, for instance, a cache, such as a shared cache, which may be coupled to local caches (examples include L1 cache, L2 cache, etc.) of processor(s) 602. Additionally, memory 604 may be or include at least one computer program product having a set (e.g., at least one) of program modules, instructions, code or the like that is/are configured to carry out functions of embodiments described herein when executed by one or more processors.

Memory 604 can store an operating system 605 and other computer programs 606, such as one or more computer programs/applications that execute to perform aspects described herein. Specifically, programs/applications can include computer readable program instructions that may be configured to carry out functions of embodiments of aspects described herein.

Examples of I/O devices 608 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device may be incorporated into the computer system as shown, though in some embodiments an I/O device may be regarded as an external device (612) coupled to the computer system through one or more I/O interfaces 610.

Computer system 600 may communicate with one or more external devices 612 via one or more I/O interfaces 610. Example external devices include a keyboard, a pointing device, a display, and/or any other devices that enable a user to interact with computer system 600. Other example external devices include any device that enables computer system 600 to communicate with one or more other computing systems or peripheral devices such as a printer. A network interface/adapter is an example I/O interface that enables computer system 600 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 610 and external devices 612 can occur across wired and/or wireless communications link(s) 611, such as Ethernet-based wired or wireless connections. Example wireless connections include cellular, Wi-Fi, Bluetooth®, proximity-based, near-field, or other types of wireless connections. More generally, communications link(s) 611 may be any appropriate wireless and/or wired communication link(s) for communicating data.

Particular external device(s) 612 may include one or more data storage devices, which may store one or more programs, one or more computer readable program instructions, and/or data, etc. Computer system 600 may include and/or be coupled to and in communication with (e.g. as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 600 may be operational with numerous other general purpose or special purpose computing system environments or configurations. Computer system 600 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g. instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method including:
determining surface shape variability of a surface of bony anatomy in a surgical region of interest;
based on the surface shape variability, generating a digital guide design of a proposed surgical guide for use against the bony anatomy during a surgical procedure, the generating determining placement and surface coverage for the proposed surgical guide and configuring the proposed surgical guide to have a corresponding footprint and guide features configured to interface with selected features of the surface of the bony anatomy;
rating the proposed surgical guide for use against the bony anatomy during the surgical procedure by performing, using the digital guide design, an automated topology-based stability assessment to produce a performance rating for the proposed surgical guide;
determining whether the performance rating for the proposed surgical guide satisfies a predefined performance rating threshold; and
performing processing based on whether the proposed surgical guide satisfies the predefined performance rating threshold.

2. The method of claim 1, further including:
obtaining input digital image data indicative of a surface topology of the surface of the bony anatomy; and
segmenting the digital image data to provide a digital representation of the surface topology of the surface, wherein the determining the surface shape variability of the surface is performed using the digital representation of the surface topology of the surface.

3. The method of claim 1, wherein the determining the surface shape variability of the surface includes:
determining surface shape variability at a plurality of discrete points of the surface; and
interpolating, from the determined surface shape variability at the plurality of discrete points of the surface, surface shape variability across at least a portion of the surface to determine the surface shape variability of the surface.
automatically identifying discrete points, of the plurality of discrete points, as having relatively high surface shape variability compared to other discrete points of the plurality of discrete points; and
selecting the features of the surface with which the guide features of the proposed surgical guide are configured to interface based on the identified discrete points.

4. The method of claim 1, further including generating and displaying on a display device a digital representation of the surface shape variability of the surface, wherein the digital representation visually indicates regions, of the bony anatomy, having higher and/or lower surface shape variability on the surface relative to other regions on the surface.

5. The method of claim 4, wherein the digital representation includes a heat map anatomy model of the bony region of interest.

6. The method of claim 1, wherein the generating the digital guide design of the proposed surgical guide uses a partially or fully-automated footprint generation process incorporating a footprint-growing algorithm that grows a footprint of the proposed surgical guide by prioritizing and selecting, as the selected features of the surface, anatomy features based on the surface shape variability to dictate (i) where to extend the footprint and (ii) the guide features to incorporate in the proposed surgical guide in order to effectively engage with the selected features of the surface of the bony anatomy.

7. The method of claim 1, wherein the generating the digital guide design of the proposed surgical guide accounts for constraints on guide properties, including guide size, wherein at least some of the constraints on guide properties include one or more of:
user-specified constraints;
avoidance of contact between the proposed surgical guide and identified portions of the bony anatomy; and
a maximum dimension of the proposed surgical guide.

8. The method of claim 1, wherein the generating the digital guide design of the proposed surgical guide selects a digital guide template as an initial digital guide design, and tweaks the initial digital guide design based on patient-specific anatomy features reflected by the surface shape variability of the bony anatomy.

9. The method of claim 8, wherein the digital guide template is selected from a collection of available digital guide templates, wherein the collection of available digital guide templates is built based on population average anatomies of discrete populations, and wherein the selected digital guide template is selected based on similarities between the patient-specific anatomy features and a population average anatomy corresponding to the selected digital guide template.

10. The method of claim 1, wherein performing the automated topology-based stability assessment performs a rigid body analysis on the digital guide design that assesses stability of the proposed surgical guide for the bony anatomy during the surgical procedure, wherein the performance rating is a function of the assessed stability.

11. The method of claim 1, wherein the automated topology-based stability assessment is based on:
(i) feedback from use of other surgical guides against other anatomies; and
(ii) similarities and/or differences between:
(a) the proposed surgical guide and the other surgical guides; and/or
(b) the bony anatomy and the other anatomies.

12. The method of claim 1, wherein the automated topology-based stability assessment applies an artificial intelligence (AI) model trained on feedback from use of other surgical guides against other anatomies to score the proposed surgical guide for use against the bony anatomy during the surgical procedure.

13. The method of claim 1, wherein the automated topology-based stability assessment determines the performance rating as a function of a plurality of surface shape variability values at a plurality of points, on the anatomy surface, with which the footprint of the proposed surgical guide is configured to engage.

14. The method of claim 1, wherein the predefined performance rating threshold is a function of the surgical procedure for which the proposed surgical guide is proposed for use.

15. The method of claim 1, wherein based on the proposed surgical guide satisfying the predefined performance rating threshold, the performing processing includes providing the digital guide design for fabrication.

16. The method of claim 15, further including physically fabricating the proposed surgical guide in accordance with the digital guide design.

17. The method of claim 1, wherein based on the proposed surgical guide not satisfying the predefined performance rating threshold, the performing processing includes modifying the digital guide design of the proposed surgical guide to modify properties for the proposed surgical guide, and re-rating the proposed surgical guide with the modified properties.

18. The method of claim 1, wherein the surgical guide is a cut guide.

19. A computer system including:
a processing circuit; and
a memory storing program instructions for execution by the processing circuit to perform a method that includes:
determining surface shape variability of a surface of bony anatomy in a surgical region of interest;
based on the surface shape variability, generating a digital guide design of a proposed surgical guide for use against the bony anatomy during a surgical procedure, the generating determining placement and surface coverage for the proposed surgical guide and configuring the proposed surgical guide to have a corresponding footprint and guide features configured to interface with selected features of the surface of the bony anatomy;
rating the proposed surgical guide for use against the bony anatomy during the surgical procedure by performing, using the digital guide design, an automated topology-based stability assessment to produce a performance rating for the proposed surgical guide;
determining whether the performance rating for the proposed surgical guide satisfies a predefined performance rating threshold; and
performing processing based on whether the proposed surgical guide satisfies the predefined performance rating threshold.

20. A computer program product including:
a computer readable storage medium storing instructions for execution by a processing circuit to perform a method that includes:
determining surface shape variability of a surface of bony anatomy in a surgical region of interest;
based on the surface shape variability, generating a digital guide design of a proposed surgical guide for use against the bony anatomy during a surgical procedure, the generating determining placement and surface coverage for the proposed surgical guide and configuring the proposed surgical guide to have a corresponding footprint and guide features configured to interface with selected features of the surface of the bony anatomy;
rating the proposed surgical guide for use against the bony anatomy during the surgical procedure by performing, using the digital guide design, an automated topology-based stability assessment to produce a performance rating for the proposed surgical guide;
determining whether the performance rating for the proposed surgical guide satisfies a predefined performance rating threshold; and
performing processing based on whether the proposed surgical guide satisfies the predefined performance rating threshold.
